(19) United States
(12) Patent Application Publication
(10) Pub. No.: US 2006/0025390 A1
(43) Pub. Date: Feb. 2, 2006

(54) Treatment of hormone allergy and related symptoms and disorders

(76) Inventor: Russell R. Roby, Austin, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 11/191,483

(22) Filed: Jul. 28, 2005

Related U.S. Application Data
(60) Provisional application No. 60/591,638, filed on Jul. 28, 2004.
(60) Provisional application No. 60/591,779, filed on Jul. 28, 2004.

(51) Int. Cl. A61K 31/56; A61K 31/57

(52) U.S. Cl. 514/170; 514/177

(57) ABSTRACT

The present disclosure provides methods and compositions for diagnosis and treatment of hormone allergy in a subject, including, without limitation, through measurement of elevated anti-hormone antibodies, e.g., IgE, IgG, and IgM. Target hormones may include estrogen and progesterone. Hormone allergy related symptoms and disorders may be treated by administration of dilute hormone solutions, such as dilute estrogen or progesterone solutions, intradermally or sublingually.

### RELATED APPLICATION

This application claims priority to U.S. Provisional Patent Application Ser. No. 60/591,638, filed Jul. 28, 2004 and entitled “TREATMENT OF AUTOIMMUNE DISEASES” and U.S. Provisional Patent Application Ser. No. 60/591,779, filed Jul. 28, 2004 and entitled “TREATMENT OF HORMONE ALLERGY ANE RELATED SYMPTOMS AND DISORDERS,” the contents of which are hereby incorporated in their entirety by reference.

### FIELD OF THE INVENTION

The present disclosure relates in general to the treatment of autoimmune diseases and in particular to treatment of autoimmune diseases with dilute hormone solutions and/or anti-immunoglobulin therapy.

### BACKGROUND

Allergies are a type of inappropriate immune reaction to an agent, normally referred to as the allergen. Many common allergens are harmless agents found outside the body, such as pollen and animal detritus, and even enzymes found in some laundry detergents. However, scientists have long suspected that allergens might also include harmless or useful agents produced by the human body itself. Because such agents are produced by the body, allergies to such agents may also be characterized as a type of autoimmune disorder. In fact, it is possible that other non-allergic autoimmune disorders to these allergens may also be present in addition to allergy and may induce some symptoms similar to allergic symptoms or may be exacerbated by various allergies.

Many autoimmune diseases may be detected by the presence of antibodies that recognize an agent produced by the human body. However, antibodies are not all the same (the four principle types are IgA, IgG, IgE and IgM) and the different types induce different responses from the immune system or are present in different areas. These antibodies, along with lymphocytes (white blood cells) can cause several types of allergic reactions.

IgE normally mediates a Type 1 allergy, often called Immediate-Type Hypersensitivity. In a Type 1 allergic reaction, allergy symptoms occur very rapidly after exposure to an allergen.

Types 2 and 3 allergies are mediated by IgG, IgM and IgA. These types of reactions are often called Delayed-Type Hypersensitivity. As this name suggests, these two types of allergic reaction take some time to develop after exposure to an allergen and in many cases will not occur absent prolonged exposure to an allergen.

Finally, Type 4 allergic reactions tend to result from direct lyphocyte reaction to an antigen that is not mediated by an antibody. This type of reaction is another sort of Delayed-Type Hypersensitivity.

All allergies may sometimes be alleviated through treatment with the allergen. In some instances the treatment may force the body to recognize that the allergen is a normal part of the environment and not a threat. The body may then be induced to become “tolerant” of the allergen. Allergens administered to induce immune tolerance are often referred to as “toleragens”.

In addition to inducing tolerance, administration of allergens may sometimes produce other alleviations of allergy symptoms. These effects are sometimes quite rapid. The reasons for non-tolerance-related alleviation of allergy symptoms by administration of an allergen are poorly understood.

In both tolerance and non-tolerance mediated relief of allergy symptoms by administration of an allergen, the dosage and mode of administration of the allergen is sometimes important. In general, administration of small amounts of the allergen tends to be more effective than administration of large amounts.

Hormone sensitivity has been previously described in the medical literature as premenstrual asthma. (Skobeloff E. M., Spivey W. H., Silverman R. A., Ekin B. A., Harchelroad F. P., Alessi T. V.: The effect of the menstrual cycle on asthma presentations in the emergency department. Arch Intern Med 1996; 156: 1837-40. Claude F.: Asthma et menstruation. Presse Med 1938; 46: 755-759; Eliasson O., Scherzer H., DeGraff A. C.: Morbidity in asthma in relation to the menstrual cycle. J Allergy Clin Immunol 1986: 77: 87-94; Chandler M. H., Schuldheisz S., Phillips B., Muse K. N.: Pre-menstrual asthma: the effect of estrogen on symptoms, pulmonary function, and beta 2-receptors. Pharmacology 1997; 17(2): 224-234.) Premenstrual asthma is a condition where premenstrual fluctuations in hormones such as estrogen and progesterone cause the exacerbation of clinical symptoms. Exacerbations of symptoms appear to occur during the premenstrual period when progesterone levels are high. Several references have been made to a possible reaction to hormones. The first report of hormonal influence on asthma symptoms appeared in a case report by Frank from 1931. (Frank, R T: The hormonal causes of pre-menstrual tension. Arch Neurol Psychiatry 1931; 26: 1053-57.) Severe aggravation of asthma symptoms in one patient were clearly linked to oral contraceptives. (Derimov, G S, Oppenheimer J: Exacerbation of premenstrual asthma caused by an oral contraceptive. Ann Allergy Asthma Immunol 1998; 81: 243-46.)

Skobeloff et. al reported a four-fold increase in the presentation of asthmatic women to the emergency department during the perimenstrual interval (days 26 to 04 of the menstrual cycle). When Skobeloff's data is superimposed over the hormone levels during the menstrual cycle, it shows that the peak emergency room visits occurred during the premenstrual period when the progesterone is highest relative to estrogen (FIG. 1).

While the impact of female sex steroids on asthma has been explored, no clear connection has emerged. Several studies have failed to show differences in spirometric measurements and airway reactivity to histamine (Weinman G G, Zacur H, Fish J E. Absence of changes in airway responsiveness during the menstrual cycle. J Allergy Clin Immunol 1987; 79:634-638) and methacholine challenge. (Pauli B D, Reid R L, Munt P W, Wigle R D, Forkert L. Influence of the menstrual cycle on airway function in asthmatic and normal subjects. Am Rev Resp Dis 1989; 140:358-362; Juniper E F, Kline P A, Roberts R S, Hargreave F E, Daniel E E. Airway responsiveness to methacholine during the natural menstrual cycle and effect of oral contraceptives. Am Rev Respir Dis 1987; 135:1039-42.)

There have been conflicting reports on the effect of estrogen on asthma in postmenopausal women. Meyers et al. reported on three cases where the estrogen supplements led to an improvement in symptoms. (Meyers J R, Sherman C B. Should supplemental estrogens be used as steroid-sparing agents in asthmatic women. Chest 1994; 106:318-19.) Improvement in PMA symptoms in females receiving exogenous estradiol was also reported by Chandler et al. (Chandler M H, Schuldheisz S, Phillips B, Muse K N. Pre-menstrual asthma: the effect of estrogen on symptoms, pulmonary function, and beta 2-receptors. Pharmacology 1997; 17(2):224-234.) However, in both of these studies, large amounts of estrogen or estradiol were supplied to the patients in normal hormone replacement therapy oral doses.

Conversely, another case study reports increased symptoms of asthma with estrogen therapy. (Collins L C, Peiris A. Bronchospasm secondary to replacement estrogen therapy. Chest 1993; 104:1300-02.) In this study high doses or estrogen were also supplied as normal oral hormone replacement therapy.

These results are not easily explained. It may be that high levels of estrogen, relative to progesterone, have a blocking effect on the allergic reaction to progesterone. In two studies by Lieberman et al. subclinical deterioration of asthma and diminished airway reactivity was demonstrated in postmenopausal women secondary to estrogen replacement. (Lieberman D, Kopernik G, Porath A, Lazer S, Heimer D. Sub-clinical worsening of bronchial asthma during estrogen replacement therapy in asthmatic postmenopausal women. Maturitas 1995; 21:153-157; Lieberman D, Kopernic G, Porath A, Levitas E, Lazer S, Heimer D. Influence of estrogen replacement therapy on airway reactivity. Respiration 1995; 62:205-208.)

While the earlier studies using histamine and methacholine challenge failed to demonstrate differences in spirometric measurements and airway reactivity, recent studies by Tan et al., established increased airway responsiveness in asthmatic females in the perimenstrual period using adenosine 5′-monophosphate (AMP) challenge. (Tan K S, McFarlane L C, Lipworth B J. Loss of normal cyclical β₂-adrenoreceptor regulation and increased premenstrual responsiveness to adenosine monophosphate in stable female asthmatic patients. Thorax 1997; 52:608-611.) Histamine and methacholine act as direct smooth muscle stimulants while AMP causes bronchoconstriction through mast cell activation. Mast cells are a key component of the bronchial allergic response.

Thus, these studies indicate that an allergy might play a role in pre-menstrual asthma in both premenopausal and postmenopausal women. A mechanism is suggested by Tan et al. in the observation that a down-regulation of lymphocyte β₂-adrenoreceptors occurs in asthmatic women following the administration of exogenous progesterone during the follicular phase. (Tan K S, McFarlane L C, Lipworth B J. Paraxial down-regulation and desensitization of β₂-adrenoreceptors by exogenous progesterone in female asthmatics. Chest 1997; 111:847-851.) This paradoxical down-regulation did not occur when estrogen was administered.

Despite these studies, little evidence has been previously developed regarding these role hormones allergy might play in pre-menstrual asthma. Schmidt et al. describe symptoms of premenstrual syndrome as abnormal response to normal concentrations of gonadal steroids. (Schmidt P J, Nieman L, Danaceau M A, Adams L F, Rubinow D R. Differential behavioral effects of gonadal steroids in women with and in those without premenstrual syndrome. NEJM 1998; 338:209-216.) Beynon et al. reported on three patients with PMA whose symptoms were decreased by the administration of intramuscular progesterone. (Beynon H L, Garbett N D, Barnes P J. Severe premenstrual exacerbation of asthma: effect of intramuscular progesterone. Lancet 1988; 2:370-72.) However, these patients continued to receive corticosteroid treatment and received high doses of progesterone at each injection (100 mg daily in two cases and 600 mg twice a week in the other). Additionally, no follow-up study with more patients or addressing the cause of the premenstrual exacerbation of asthma was ever conducted.

Overall, there has been little investigation into the systemic manifestations or treatment of hormone allergy. Additionally, little research has been conducted regarding the mode of action of such hormone allergies.

### SUMMARY

The disclosure relates to methods of treating hormone allergy, including in some embodiments related symptoms or disorders, by administering a dilute hormone solution to a subject having a hormone allergy.

In a specific embodiment, the method may include administering 0.5 mg of progesterone intradermally or sublingually. In another embodiment it may include administering 0.4 mg of estrogen intradermally or sublingually. In some example embodiments, the disclosure provides a composition for attenuating an IgE-mediated hormone immune response in a subject having a hormone allergy including about 10⁻¹⁰ milligrams to about 10⁻¹ milligrams of the hormone and a pharmaceutically acceptable additive.

In some embodiments, the treated symptoms or disorders may include irritation or pain in the neck, nose, eyes, throat, ear, skin, back, hips, upper extremities and lower extremities, headache, including migraine headaches, shortness of breath, including asthma, arthritis, infertility, fibromyalgia, chronic fatigue syndrome, and multiple sclerosis.

The disclosure also includes a method of diagnosing a hormone allergy in a subject. In order to make such a diagnosis the level of an anti-hormone antibody in blood serum of the subject may be determined. This may then be compared to the normal level of an anti-hormone antibody in the blood sera of individuals who do not have a hormone allergy. Treatment benefits may also be objectively measured by comparing pre-treatment antibody levels with post-treatment levels. A statistically significant elevated level of anti-hormone antibody in the blood serum of the subject as compared to the normal level of anti-hormone antibody in the blood sera of individuals who do not have hormone allergy or a sharp fall of anti-hormone antibody after treatment may be indicative of hormone allergy in the subject.

In specific embodiments, the anti-hormone antibody may be an anti-progesterone antibody or an anti-estrogen antibody. The antibody may also be an immunoglobin, e.g., IgE. Antibody levels are determined by ELISA assay.

Some embodiments of the disclosure may reveal high levels of IgE antibodies against progesterone and estrogen, and establish hormone allergy.

### BRIEF DESCRIPTION OF THE DRAWINGS

Some embodiments of the disclosure may be understood by referring, in part, to the description herein and the following drawings.

FIG. 1 illustrates the number of female asthmatic patient visits to an emergency department during the menstrual cycle.

FIG. 2 illustrates the response of various hormone allergy symptoms to treatment with dilute hormone solution.

FIG. 3 illustrates responsiveness of asthma patients to dilute hormone solution treatment by percentage of asthma relief experienced.

FIG. 4 illustrates Mean+S.D. of ELISA Units for IgG or IgM against progesterone in 288 controls and 270 patients with possible hormone allergy.

FIG. 5 illustrates the percentage of elevation in IgG, IgM and IgG+IgM antibodies against progesterone controls and patients with possible hormone allergy at the cutoff point of 21 ELISA units.

FIG. 6 illustrates Mean+S.D. of ELISA units for IgE against progesterone controls and patients with possible hormone allergy.

FIG. 7 illustrates the percentage of elevation in IgE antibodies in controls and patients with possible hormone allergy at cutoff point of 22 ELISA units.

FIG. 8 illustrates Mean+S.D. of ELISA units for IgE against estrogen in controls and patients with possible hormone allergy.

FIG. 9 illustrates the percentage of elevation in IgE antibodies against estrogen in controls and patients with possible hormone allergy at cutoff point of 19 ELISA units.

FIG. 10 illustrates hormone as a hapten reacts with carrier protein to stimulate TH2 activation and production of IgE against hormones or their metabolites. The IgE antibodies bind to mast cells, including the release of histamine and other mediators, resulting in systemic anaphylactic reaction. Hapten carrier can stimulate lymphocytes inducing lymphoblast transformation resulting in Type IV allergic reaction.

### DETAILED DESCRIPTION

The present disclosure relates to methods and compositions for treating hormone allergies and their related symptoms and disorders. It also includes methods for diagnosing hormone allergies.

In a specific embodiment, the disclosure includes dilute hormones for the treatment of symptoms and disorders related to hormone allergy. Normally, the dilute hormone used for treatment is the hormone to which the subject is allergic. Thus, in female subjects the hormone is commonly progesterone or estrogen (general references to “progesterone” or “estrogen” herein are intended to include any analogs thereof functional in the methods and compositions of the present disclosure).

The amount of hormone administered may be the minimal amount needed to alleviate the relevant symptoms. Thus, the appropriate amount may be determined simply by administering to the subject increasing amounts of hormone until alleviation of the symptoms is achieved. While it is possible to administer to the subject an amount of hormone greater than the minimal amount able to achieve alleviation of symptoms, in a specific embodiment, only the minimal amount is administered. Additionally, the minimal amount of hormone able to alleviate symptoms may change during the course of treatment. Such change in minimal dose may also be determined by administering to the subject increasing amounts of hormone until alleviation is achieved. Alternatively, a small dose of hormone may be chosen for administration to most subjects. Such dose may be previously determined to be effective in a certain percentage of subjects.

All compositions of the present invention may be administered with or in a pharmaceutically-acceptable additive. Additives may be selected from the group consisting of carriers, excipients, and diluents. Suitable carriers include buffers such as phosphoric acid, citric acid and other organic acids; antioxidants such as ascorbic acid; low-molecular weight polypeptides; proteins such as serum albumin, gelatin and immunoglobulin; hydrophilic polymers such as polyvinylpyrrolidone; amino acids such as glycine, glutamine, arginine or lysine; monosaccharides such as mannose or dextrin, disaccharides, other carbohydrates; chelating factors such as EDTA; metal ions such as zinc, cobalt or copper; sugar alcohols such as mannitol or sorbitol; salt-forming counter ions such as sodium; and/or non-ionic surfactants such as Tween, Pluronic or polyethylene glycol (PEG). Excipients and diluents may be selected from the group consisting of magnesium stearate, calcium carbonate, starch-gelatin paste, talc, aluminum salt, phenoxyethyl ethanol, water, physiological salt solution, lactose, dextrose, sucrose, sorbitol, mannitol, calcium silicate, cellulose, methyl cellulose, amorphous cellulose, polyvinylpyrolidone, metylhydroxy bezoate, propylhydroxybezoate, and a mineral oil. Other optional components, e.g., stabilizers, buffers, preservatives, flavorings, excipients and the like, may be added.

An anti-hormone allergy compound (e.g., hormone and/or immunoglobulin) may be formulated in any physiologically acceptable carrier. In a specific embodiment, the carrier may be a liquid carrier including an alcohol and oil, or including a saline solution. The volume of carrier may vary, but it may be selected so as to allow delivery of the desired amount of hormone in a small volume, such as one milliliter or less, specifically one hundred microliters. The carrier and volume may be selected based on a variety of factors, including the mode of delivery, the form or concentration in which the hormone is supplied before formulation, and the ability to administer a precise amount of hormone. Although the initial hormone may be supplied in any form, in certain embodiments it may be obtained as an injectable, solubilized hormone that is then further diluted in the carrier.

An anti-hormone allergy compound (e.g., hormone and/or immunoglobulin) may be administered through any effective mode including, without limitation, sublingual administration and intradermal injection. Other possible routes include oral administration, paraenteral administration, intradermal injection, subcutaneous injection, intrathyroid injection, and intravenous injection, intranasal, transdermal, transconjunctival, or aerosol mist through any orofice or through the skin. The invention additionally contemplates thyroid hormone delivery using a suitable gene therapy vector.

Compositions of the invention may have a form selected from the group consisting of ingestible tablet, buccal tablet, troches, capsule, elixir, suspension, syrup, wafer, pill, granule, powder, cachet, emulsion, liquid, aerosol, soft or hard gelatin capsule, sterilized liquid for injection, sterilized powder and the like.

In a more specific embodiment, the hormone may be administered to an adult human in an amount between 0.01 and 10 mg, more specifically 0.1 and 1 mg. Still more specifically, 0.5 mg of progesterone or 0.4 mg of estrogen may be administered.

The hormone may be formulated in any physiologically acceptable carrier. In a specific embodiment, the carrier may be a liquid carrier including an alcohol and oil, or including a saline solution. The volume of carrier may vary, but it may be selected so as to allow delivery of the desired amount of hormone in a small volume, such as 1 ml or less, specifically 0.1 ml. The carrier and volume may be selected based on a variety of factors, including the mode of delivery, the form or concentration in which the hormone is supplied before formulation, and the ability to administer a precise amount of hormone. Although the initial hormone may be supplied in any form, in certain embodiments it may be obtained as an injectable, solubilized hormone that is then further diluted in the carrier.

The hormone may be administered through any effective mode. According to some specific example embodiments, administration may specifically be sublingual or by intradermal injection.

The treated symptoms and disorders related to hormone allergy may include irritation or pain in the neck, nose, eyes, throat, ear, skin, back, hips, upper extremities and lower extremities, headache, including migraine headaches, shortness of breath, including asthma, arthritis, infertility, fibromyalgia, chronic fatigue syndrome and multiple sclerosis. In some embodiments, a hormone allergy may include an allergy to progesterone and/or estrogen, e.g., in female subjects.

In specific embodiments, methods of treating hormone allergy include administration of dilute hormone compositions as described above to alleviate the symptoms or disorders also described above.

Certain embodiments of the present disclosure are additionally related to methods of diagnosing hormone allergy in subjects, e.g., female subjects. Because the presence of IgE is required for a Type 1 allergic reaction, detection of elevated of anti-hormone IgE is indicative of a Type 1 hormone allergy. Presence of IgG, IgM or IgA may be indicative of a Type 2 or 3 hormone allergy. An assay for such Igs may be particularly useful in subjects exhibiting the symptoms or disorders described herein. Detection of elevated anti-hormone immunoglobin (Ig) provides a clue as to which hormone may be responsible for the symptoms or disorder, thus guiding treatment. Failure to detect elevated levels of anti-hormone Ig may indicate that the symptoms or disorder are caused by something other than hormone allergy, such as a different autoimmune disorder. In some embodiments, a hormone allergy diagnosis is made before or concurrently with other types of diagnostic assays.

In specific embodiments diagnosis may focus on detection of anti-hormone IgE because of its role in rapid allergic responses.

In subjects that exhibit elevated anti-hormone antibodies, e.g., IgG, IgM, and/or IgE, as well as subjects that have inconclusive results or do not exhibit elevated antibody levels, a decrease in anti-hormone antibodies, e.g., IgG, IgM, and/or IgE, after treatment may still be indicative of a hormone allergy. This may be particularly true if the subject additionally exhibits improvement in a hormone allergy-related symptom or disease after treatment. Thus, although many subjects with hormone allergy may be identified by high levels of anti-hormone antibodies, this method may not be suitable for all subjects. For example, subjects who produce low amounts of antibodies overall as compared to normal subjects may require diagnosis by this second method.

The present disclosure further relates to a method of treating IgE-mediated autoimmune diseases and allergies to bodily produced substances through administration of an anti-IgE treatment.

In certain embodiments, the disease or allergy may include hormone allergy, pain in the neck, nose, eyes, throat, ear, skin, back, hips, upper extremities and lower extremities, headache, including migraine headaches, shortness of breath, including asthma, arthritis, infertility, fibromyalgia, chronic fatigue syndrome, and multiple sclerosis.

The anti-IgE treatment may be any treatment sufficient to reduce or inhibit IgE levels in a manner to alleviate symptoms of the disease or allergy. In more specific embodiments, the treatment may include an anti-IgE antibody, such as a humanized antibody. For example, it may include the antibodies currently produced by Genentech, C A and marketed as Xolair, or by Tanox, T X currently labeled as TNX-901.

Other anti-IgE antibodies may be developed and testing using methods known to the art to determine if they are more effective against hormone allergy or in the relief of any particular disease or symptom, such as one of those described above.

The anti-IgE antibodies may be administered in any appropriate manner or formulation. In specific embodiments, they may be administered by injection in a pharmaceutically acceptable carrier, such as a saline-based carrier.

The anti-IgE antibodies may be administered continuously or intermittently, but in specific embodiments, they may be administered at the onset of symptoms, or at another time-appropriate juncture, such as before conception is desired in infertile patients.

According to some embodiments of the disclosure, formulations are prepared and administered such that subjects experience relief quickly. For example, relief from at least one hormone allergy symptom may be experienced in less than about 30 minutes, less than about 20 minutes, less than about 10 minutes, less than about five minutes, less than about one minute, or less than about 30 seconds.

Without being limited to any particular mechanism of action, when IgG, IgE, and IgM were measured against progesterone, the highest levels were detected first against 17-hydroxyprogesterone, followed by progesterone, hydrocortisone, and then estrogen (Table 4). And where a patient demonstrated the highest levels of IgG, IgM or IgE antibodies against estrogen, these antibodies were detected significantly against 17-hydroxyprogesterone, followed by progesterone and hydrocortisone. Similar to the above, no antibodies were detected against cholesterol (Table 5) This lack of detection of antibodies against the parent compound of steroids (cholesterol) is the best indication of antibody specificity against certain chemical structures of steroids. Since in three different patients IgG, IgM and IgE isotype antibodies were detected first at the highest level against 17-□-hydroxytestosterone, then against progesterone and hydrocortisone, these antibodies have an apparent binding affinity to OH-group at C₁₁ of 17-□-hydroxyprogesterone, estrogen or estrogen metabolites, including 2-HE, 4-HE and 16-□-HE. In relation levels of these metabolites, a lot may be learned from carcinogenesis studies. Most researchers agree that 4-HE and 16-□-HE can induce tumors in experimental animals. Researchers discovered that an enzyme known as cytochrome P-450 1B1 converts 17-□-estradiol to 4-HE. Further, numerous enzymes can change 4-HE into compounds called 3,4-semiquinones and 3,4-quinones, which damages DNA and leads to tumor development. In relation to 16-□-HE, epidemiological studies with 85 women showed that those who had lower 2-HE to 16-□-HE ratio in urine were more likely to go on to develop breast cancer than were women in whom that ratio was more in favor of 2-HE. Despite its low levels in the body, estrogen may be present in sufficient quantities to produce enough by-products to raise concern. Estrogen may also be synthesized directly in cells in target tissue, far exceeding the amount of the hormone in blood. Since the tissue itself makes estrogen, there may be enough present to make high levels of estrogen metabolism to make protein and DNA adducts. While the protein-hormone metabolite adducts may induce anti-hormone and anti-tissue antibodies, the DNA-hormone adducts may trigger DNA damage and cancer.

The inhibition and cross-reaction of different sera containing IgG, IgM or IgE antibodies against estrogen and 17-□-hydroxyprogesterone with different hormone compounds presented in this study is further indication that hormone metabolites play a critical role in antibody production in these experiments (Tables 3, 4).

Detection of high levels of IgG, IgM and IgE antibodies in the sera from a subgroup of patients and its inhibition by steroids-bound to BSA is an indication for reactions of steroids as haptens with body proteins and production of antibodies against this new antigenic epitope(s). And since the highest levels of antibodies were detected against 17-□-hydroxyprogesterone, we believe that OH-group at C₁₁ plays a role in the formation of this new antigenic material in a subgroup of patients with endocrine allergy. Data presented here clearly demonstrates that antibody formation against small molecules such as steroids is possible.

Estrogen and progesterone may be successfully bound to BSA or peroxidase. In fact, for these preparations, steroids were modified by the binding of amino acids of carrier proteins to OH-group of C₁₁. Therefore, when these hormones, whether from endogenous or exogenous source, rise to certain levels, hormone metabolizing enzymes get activated and form different metabolites. In individuals with specific HLA types and low or high levels of hormone metabolizing enzymes (cytochrome P150 1B1 and others) these metabolites bind to tissue proteins and induce immune response in a form of IgG, IgM and/or IgE production.

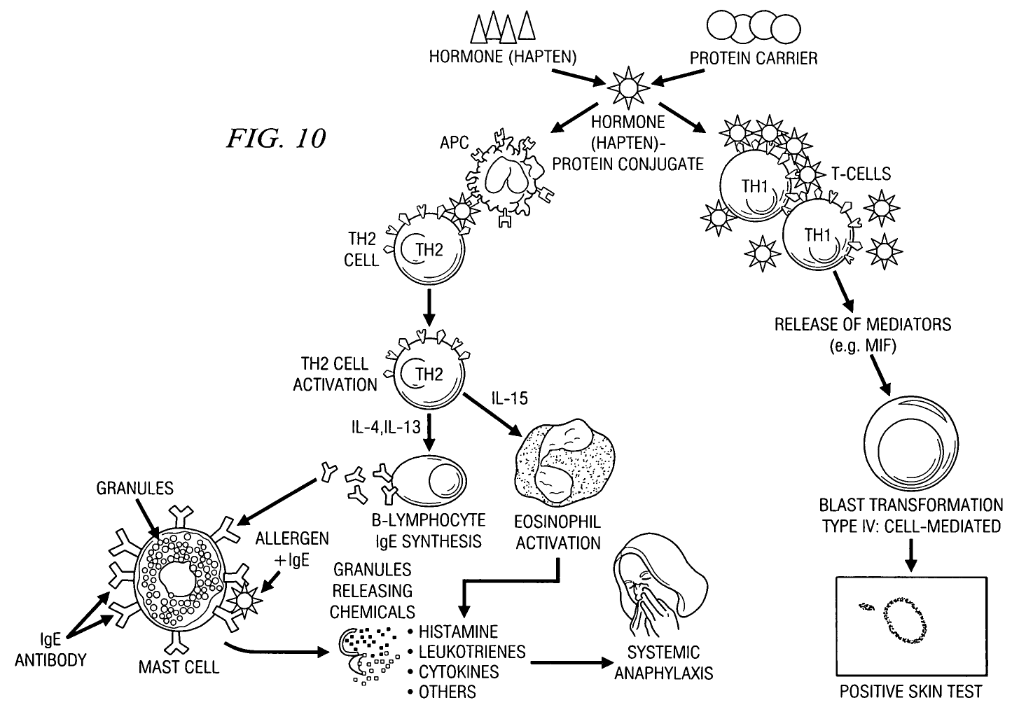

It is possible that several mechanisms of actions are at work in the process of hormone allergy. One of these possibilities may be that estrogen, progesterone and their metabolites, after binding to different proteins, may act as antigens and promote T-helper-2 cell development, thereby regulating IgE or other antibody synthesis. The binding of these antibodies to mast cells with their corresponding antigens (hormones or metabolites) induce mast cell or basophil degranulation. This reaction leads to histamine release, TH2 cytokine and leukotriene secretion, resulting in systemic anaphylaxis in the form of allergy or asthma (FIG. 10).

A second possibility is that after hormones bind to blood proteins, different lymphocytes will react to this complex and induce lymphocyte proliferation and cytokine production, resulting in Type IV allergic reaction or delayed-type hypersensitivity. These mechanisms are summarized in FIG. 10.

According to some embodiments, many disorders may be effectively treated by determining the presence of hormone allergy and use of hormones as antigens to diminish symptoms by desensitization.

### EXAMPLES

The following examples are provided to further explain specific examples of the disclosure. They are not intended to represent all aspects of the disclosure in its entirety. Variations will be apparent to one skilled in the art.

### Example 1

### Anti-Hormone Antibodies in Patients Responding to Hormone Allergy Treatment

Patients undergoing treatment for hormone allergy involving intradermal or sublingual administration of dilute progesterone were tested by prick test for the presence of anti-progesterone IgG, IgM, or IgE antibodies using an ELISA assay. Blood samples obtained from 271 unselected patients were tested for anti-progesterone IgG and IgM. In these patients, 52% exhibited high IgG or IgM antibodies. Blood samples from 88 different patients and 88 healthy individuals were tested for anti-progesterone IgE and anti-estrogen IgE. Anti-progesterone IgE antibodies were high in 41% of patients tested. Anti-estrogen IgE antibodies were high in 86% of patients tested.

Over half of all patients who reported relief of at least 60% of the twelve hormone allergy symptoms described further in Example 2 when administered dilute progesterone additionally had high levels of anti-progesterone IgG, IgM and IgE.

This indicates that in a significant number of patients may have a Type 1 allergic reaction to progesterone. This type of reaction is very rapid and often quite dangerous, even when compared to other types of allergic reactions. Because it is so rapid, it does not require long-term exposure to an antigen or exposure to a large amount of antigen.

Additionally, the presence of elevated IgG, and IgM in hormone allergy patients indicated that some responses may also be Type 2 or 3 allergic responses.

Finally, the absence of anti-progesterone antibodies in several hormone allergy patients may indicate a Type 4 allergic response.

### Example 2

### Treatment of Hormone Allergy Symptoms

Patients experiencing several hormone allergy symptoms experienced relief when administered dilute doses of progesterone either sublingually or intradermally. Further, as compared to several other antigens, progesterone proved most effective in producing these effects. Additionally, relief is experienced very quickly in most patients, further suggesting that their progesterone allergy might be a Type 1 allergic reaction.

The symptoms measured were irritation or pain in the neck, nose, eyes, throat, ear, skin, back, hips, upper extremities and lower extremities, headache, and shortness of breath. The patients were administered 0.5 mg of progesterone intradermally.

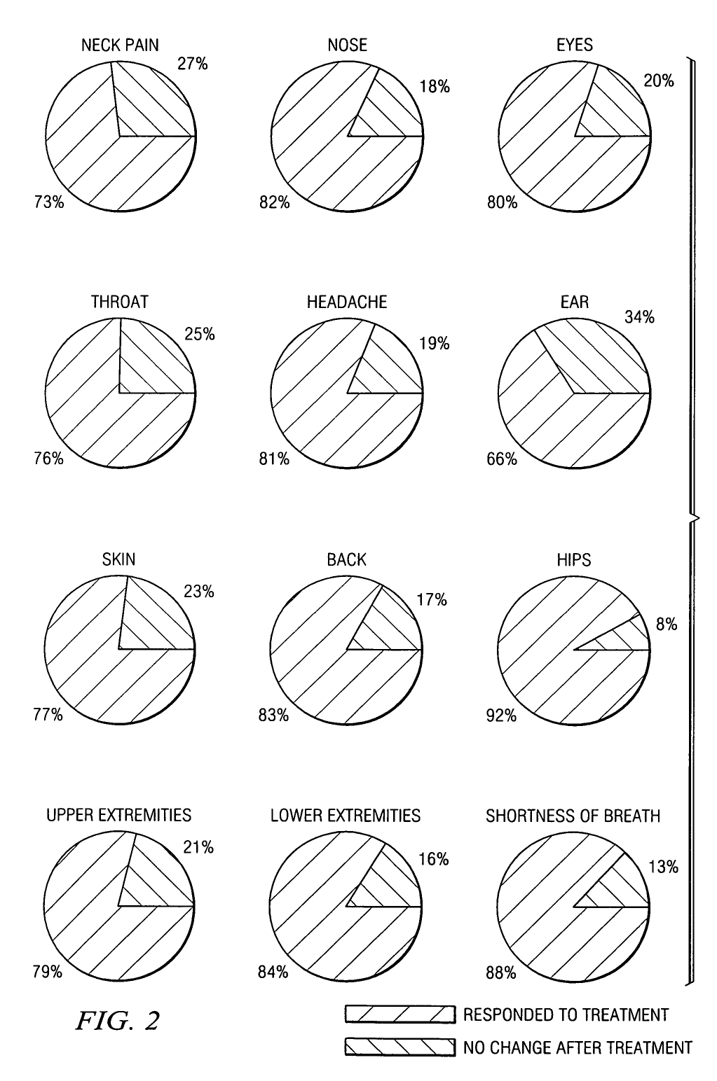

The results of these tests are summarized in Tables 1 and 2 and FIG. 2.

TABLE 1
Clinical Responses to Intraderrnal
Administration of Progesterone

| | | | % of | |
|---|---|---|---|---|
| | | # of | Patients | % Improvement |
| | # of | Responding | who | in Responding |
| | Patients | Patients | Respond | Patients |
| Neck | 144 | 104 | 72% | 62% |
| Nose | 109 | 89 | 82% | 63% |
| Eyes | 90 | 73 | 81% | 57% |
| Throat | 92 | 70 | 76% | 64% |
| Headache | 103 | 84 | 82% | 69% |
| Ear | 35 | 23 | 66% | 25% |
| Skin | 34 | 27 | 79% | 74% |
| Back | 57 | 47 | 82% | 72% |
| Hips | 26 | 24 | 92% | 79% |
| Lower | 61 | 48 | 79% | 76% |
| Extremity | | | | |
| Upper | 50 | 40 | 80% | 75% |
| Extremity | | | | |
| Shortness | 142 | 124 | 87% | 67% |
| of Breath | | | | |

Patients were designated as responding patients in Table 1 if they experienced any relief of the indicated symptom. Patients were also asked to estimate the amount of improvement in the symptom, an average of which is

TABLE 2
Probability of Results due to Random
Effects

| | | | | Probability |
|---|---|---|---|---|
| | # of | | | of + |
| | Patients | # of | # of non- | Results due |
| | with | Responding | Responding | to Random |
| | Symptom | Patients | Patients | Effects |
| Neck | 146 | 106 | 40 | <= 0.0001% |
| Nose | 112 | 92 | 20 | <= 0.0001% |
| Eyes | 94 | 75 | 19 | <= 0.0001% |
| Throat | 94 | 71 | 23 | <= 0.0001% |
| Headache | 105 | 85 | 20 | <= 0.0001% |
| Ear | 35 | 23 | 12 | <= 5% |
| Skin | 35 | 27 | 8 | <= 1% |
| Back | 58 | 48 | 10 | <= 0.0001% |
| Hips | 26 | 24 | 2 | <= 0.0001% |
| Lower | 51 | 43 | 8 | <= 0.0001% |
| Extremity | | | | |
| Upper | 63 | 50 | 13 | <= 0.0001% |
| Extremity | | | | |
| Shortness | | | | |
| of Breath | 144 | 126 | 18 | <= 0.0001% |

In Table 2 the probability that positive results (i.e. patients responding to treatment) were due to random effects was calculated using standard statistical analysis of patient tests. It was assumed that if the treatment has no effect, there is a 50% chance that a patient will indicate a positive response and a 50% chance that a patient will indicate a negative response. From this assumption, coupled with the actual data, one may calculate the probability that positive responses are due to an actual effect of the treatment and not simply random effects. In general, results with a less than 1% chance of being random are considered highly significant. Results with a less than 5% chance of being random are normally conclusively labeled as nonrandom. Thus, the results observed for most of the symptoms in Table 2 are highly significant, with only the results for ear irritation and pain falling merely in the nonrandom category.

The results of Table 2 are depicted graphically in FIG. 2.

Further tests have shown that many patients whose symptoms are not relieved using progesterone treatment do experience relief when administered 0.4 mg of estrogen intradermally.

### Example 3

### Hormone Allergy in Female Asthmatics

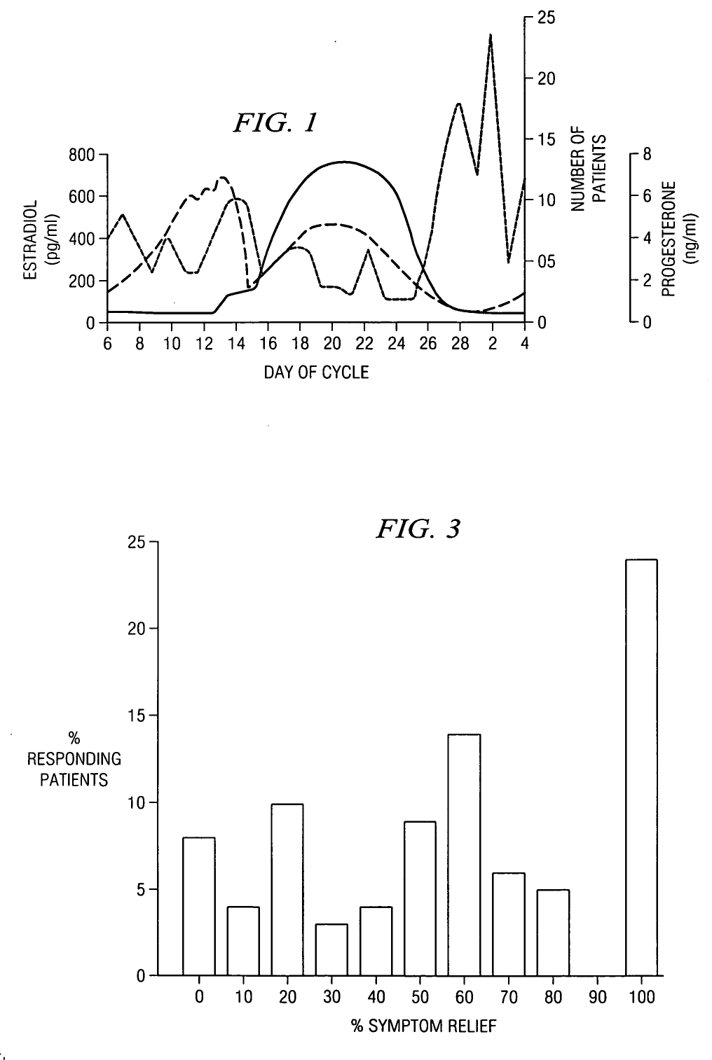

FIG. 1 shows that hospital admissions for asthma symptoms are highest during the perimenstrual interval (days 26 to 04) of the menstrual cycle when progesterone levels are highest relative to estrogen levels. Based on the results presented in Examples 1 and 2, particularly those related to relief of shortness of breath, coupled with this increase in hospital admissions, it appears that there may be a relationship between premenstrual asthma and hormone allergy.

To test this hypothesis, 142 women with acute asthma were asked to evaluate the severity of their symptoms using an eleven point scale. Ten was assigned to the most severe symptoms, while 0 was assigned to an absence of symptoms. Immediately after assessing their symptoms on this scale, patents were administered 0.5 mg of progesterone by intradermal injection.

Approximately 15 to 30 seconds after the injection was administered, each patient was asked if her symptoms were worse, better, or unchanged. Each patient was also asked to rate her present symptoms on the same 0 to 10 scale.

The results of these tests are shown in FIG. 3. FIG. 3 shows the % of patients responding to treatment by % of symptom relief reported. Of the 142 patients tested 129 (91%) positively responded to the treatment. Using the random effects calculations described in Example 2, this correlates to a less than 0.1% chance that the results are random. Thus the results may be considered highly significant.

Of the 129 patients who experienced symptom relief, 40 (28%) reported 100% relief of their asthma symptoms. Using the 0 to 10 scale described above, the average symptomatic relief for all patients (those responding and those not responding to treatment) was 3.4 points. The average symptomatic relief for those who reported some response to the treatment was 5 points. Response change relative to the maximum individual patient score averaged a 62% reduction in symptoms.

This indicates that hormone allergy may play a role in premenstrual asthma.

### Example 4

### Double-Blind, Randomized, Placebo-Controlled Study of Hormone Allergy Treatment in Adult Female Patients with Asthma, Pain, Migraine Headaches, Infertility, Fibromyalgia, Chronic Fatigue Syndrome, or Multiple Sclerosis

As described above in Examples 1 to 3, a number of patients with various symptoms exhibit anti-progesterone or anti-estrogen antibodies that might indicate allergies to these hormones. In addition, many of these patients show a rapid reduction in symptoms in response to administration of small doses of progesterone or estrogen sublingually or intradermally (doses are normally 0.5 mg or 0.4 mg, respectively).

Hormone allergy treatment of patients may also be used to treat other symptoms or disorders experienced by these patients. Specifically, patients with arthritis, asthma, pain, migraine headaches, fibromyalgia, chronic fatigue syndrome and multiple sclerosis showed a reduction of disorder-related symptoms by at least 60% in over half of all patients tested. Further, of 16 infertile patients receiving hormone allergy treatment, two became pregnant and one delivered a healthy baby.

Additionally, anti-hormone antibodies were found in several multiple sclerosis patients whose multiple sclerosis-related symptoms also improved during hormone allergy treatment. Although multiple sclerosis is directly mediated by anti-myelin hormones, it appears that the hypersensitivity reaction involved in hormone allergy may aggravate the disorder. Given that various mediators of the immune response are known to be shared between the immune reaction giving rise to multiple sclerosis and the immune reaction giving rise to allergy, this aggravation of multiple sclerosis symptoms by hormone allergy appears plausible. It is possible that shared components of the immune reaction may also explain some of the positive results seen when hormone allergy is treated in patients with other disorders mediated by the immune system, such as arthritis and asthma and also possibly infertility.

The beneficial effects of hormone allergy treatment through administration of dilute amounts of progesterone or estrogen may be further confirmed in a double-blind clinical trial.

Potential subjects may be asked to rate their symptoms on a scale of 0 to 10, with 0 representing no symptoms and 10 representing the worse symptoms the patient experiences. Subjects rating their symptoms at level 6 or higher may be selected for the study. Patients selected may then be administered 0.5 mg of progesterone sublingually. If the patient indicates a decrease in symptom level on the 0 to 10 scale by at least 50% within 60 seconds, the patient may be administered progesterone during the study. If not, the patient may be similarly tested with 0.4 mg of estrogen administered sublingually. If the patient responds to the estrogen, then the patient may receive estrogen during the study.

Patients who qualify under the initial progesterone or estrogen tests may then be tested to see if they have high levels of anti-progesterone and/or anti-estrogen IgE. Only those exhibiting high levels of at least one of these two antibodies may be included in the trial.

Next, patients may be asked whether they have a history of regularly experiencing symptoms higher than 5 on the scale of 0 to 10. Those who do not may be excluded from the trial.

Other relevant assessments of the patients' disorder severity and symptoms may also be made at the beginning of the trial, before hormone treatment.

Patients enrolled in the trial may then receive sublingual doses of either 0.5 mg of progesterone or 0.4 mg of estrogen twice daily for 90 days. Control subjects may receive a placebo containing no hormone. Enough patients may be enrolled for each disorder group to give statistically significant results. Similarly, enough patients may be included in the control groups for each disorder group to give statistically significant results. For example, 100 subjects may be enrolled in the trial for each of the eight disorders. Of these subjects, 90% may be assigned to the test group and 10% may be assigned to the control group.

Presence of the relevant disorder may be determined based on a previous diagnosis by a physician. Infertility may be defined as having undergone one or more failed in vitro fertilization attempts or two or more spontaneous abortions. Patients may be females between the ages of 20 and 70.

At the end of the 90 days, patient anti-hormone IgE levels may be retested. Disorder symptoms may also be reassessed. Usage of other medications to treat the patients' symptoms or disorders may also be monitored throughout or at the end of the study to determine if the hormone treatments cause any alterations in the patients' usage of other medications.

Standard statistical methods may be used to analyze study results, such as changes in anti-hormone IgE levels, symptom ratings, symptoms and medication usage.

### Example 5

### Hormone Sample Preparations

Hormone samples may be prepared in any way suitable for the intended administration route. For administration through intradermal injection, samples may be prepared as they were in the studies of Examples 2 and 3. Specifically, the progesterone was formulated from commercially available injectable progesterone. Each ml of the final formulation contained 50 mg of progesterone and 10% benzyl alcohol as a preservative in sesame oil. 0.1 ml was administered per patient intradermally. Estrogen samples were similarly prepared.

For sublingual administration, such as described in Example 4, progesterone commercially available for injection at a concentration of 50 mg/ml may be diluted 10:1 in normal saline to arrive at a final concentration of 0.5 mg/ml. Similarly, commercially available injectable estrogen may be diluted from the supplied concentration of 40 mg/ml in normal saline to arrive at a concentration of 0.4 mg/ml. These formulations may be administered in 1 ml doses.

### Example 6

### Blood

Hormone levels are examined as part of our routine work-up of all adult allergy patients. When prick and sublingual tests with hormones resulted in changes in symptoms we began to test for hormone antibodies. Over a three year period, 368 female patients were tested for hormone antibodies.

For the first two years, only IgM and IgG antibodies to progesterone were tested since that was the hormone most commonly associated with symptom changes when used as a test antigen. Blood samples were taken from 270 female patients who experienced a change in symptoms associated with their menstrual cycle. The women were 24-47 years of age. Blood samples were obtained from 288 healthy control subjects by a commercial lab (Immunosciences Lab., Inc., Beverly Hills, Calif.). During the last year IgE against estrogen and progesterone was checked using 288 healthy control female subjects, 32 healthy patients as controls and 98 patients who noted perimenstrual symptom changes.

### Example 7

### Hormones, Antibodies and Reagents

Estradiol-BSA and progesterone-BSA, hydrocortisone, BSA, cholesterol, human serum albumin (HSA), bovine serum albumin (BSA), phosphate buffer saline (PBS) and substrate (PNPP) were purchased from Sigma Chemicals (St. Louis, Mo., USA).

Alkaline phosphatase-labeled goat anti-human IgG, IgM and IgE were purchased from KPL (Gaithersburg, Md., USA).

### Example 8

### ELISA for Estrogen and Progesterone Antibody

ELISA. Enzyme-linked immunosorbent assay (ELISA) was used for testing antibodies against estrogen and progesterone in the sera of patients with premenstrual asthma and with control subjects. Different rows of microtiter plates (Costar) were coated either with 100 μl of BSA concentration of 10 μg/mL or 100 μl of estrogen-BSA or progesterone-BSA optimal concentration of 10 μg/mL in 0.1 m carbonate-bicarbonate buffer (pH 9.5). Plates were incubated overnight at 4° C. and then washed three times with 200 ml of Tris-buffered saline (TBS) containing 0.05% Tween 20, pH 7.4. The non-specific binding of immunoglobulins (Igs) was prevented by adding a mixture of 1.5% bovine serum albumin (BSA) and 1.5% gelatin in TBS and then incubating this mixture for 2 h at room temperature and then overnight at 4° C. Plates were washed with PBS-Tween 20 and then 100 μl of control or patient's serum was added to duplicate wells coated either with BSA alone or with estrogen or progesterone bound to BSA. The optimal dilution of serum was determined by checkerboard dilution and found to be 1:100 for IgG and IgM and 1:2 for IgE. Plates were incubated for 2 h (for IgG and IgM) and overnight (for IgE), and then washed four times with PBS-Tween 20. Alkaline-phosphatase-conjugated goat anti-human IgG, IgM or IgE F(ab′)₂ fragment at optimal dilution of 1:700 (IgG); 1:500 (IgM) and 1:250 (IgE) was added to corresponding wells. The plates were then incubated for an additional 2 h at room temperature. After washing five times with TBS-Tween buffer, the enzyme reaction was started by adding 100 μl of para-nitrophenylphosphate in 0.1 mL of diethanolamine buffer (1 mg/ml) containing 1 mM MgCl₂ and sodium azide, pH 9.8. The reaction was stopped 45 minutes later with 50 μl of 1 N NaOH. The optical density was read at 405 nm (OD₄₀₅) with a microtiter reader. Optical densities coated with BSA alone were not more than 0.2. However, this non-specific O.D. was subtracted from wells coated with estrogen or progesterone bound to BSA.

In the next step, for construction of standard curve and conversion of optical densities to ELISA values, the following three calibrators were used:

Calibrator I—Serum from patient with no known allergy giving optical density of 0.2-0.4 at 405 nm when serum was diluted at 1:100 for IgG or IgM and 1:2 for IgE antibodies. This control was assigned an ELISA value of 10.

Calibrator II—Serum from patient with mild hormone allergy giving optical density of 0.41-1.0 when diluted at 1:100 for IgG or IgM and 1:2 for IgE antibodies. This control was assigned an ELISA value of 20.

Calibrator III—Serum from patient with severe hormone allergy giving optical density greater than 1.0 when diluted at 1:100 for IgG or IgM and 1:2 for IgE antibodies. This control was assigned an ELISA value of 80.

Negative control serum—Serum from healthy individual, which, at similar dilutions, will not give an O.D. greater than 0.3 when measured at 405 nm.

Positive control serum—Serum from patient with hormone allergy, which, at similar dilutions, will not give an O.D. greater than 0.7 when measured at 405 nm.

These calibrations were used to:

Construct a curve by plotting the mean absorbance obtained for each calibrator against its concentration on a linear graph paper, with absorbance on the vertical (y) axis and concentration on the horizontal (x) axis.

Using the mean absorbance value for each control and unknown samples, determine the corresponding concentration of gluten antibody from the standard curve.

$$\frac{ELISA Value}{of Test Specimens} = \frac{Value of Calibrator \times Absorbance of Test Specimen}{Absorbance of Calibrator}$$

This calculation is done automatically by the ELISA reader.

### Example 9

### Specificity of Hormone Antibodies

Absorption of sera with specific and non-specific antigens was used to demonstrate that these anti-hormone antibodies are specific. For this, microtiter plates were coated with hormones and blocked by the addition of 2% BSA in PBST. 100 μl of serum diluent buffer was added to all wells. Then estrogen, estrogen-BSA, progesterone-BSA, BSA, myelin basic protein (MBP), and human serum albumin (HSA) starting at concentration of 1 mg/mL was added to the second rows of 1-8 strips and titered down the column in ½ log dilution. After 60 minutes incubation 100 μl serum anti-estrogen or anti-progesterone was added to all wells. Addition of enzyme-labeled second antibody after incubation and washing resulted in color development, which was measured at 405 nm. Results were calculated as percentage of inhibition in antigen-antibody reaction.

### Example 10

### Reaction of 17-α-hydroxyprogesterone- and Estrogen-Binding Antibodies to Other Hormones

The ELISA inhibition method was used to determine specificity and cross-reactivity between different hormones and to ensure the degree of human IgG, IgM and IgE estrogen or progesterone binding to estrogen or progesterone in solid phase. Different microtiter plates were coated with optimal concentration (100 μg/mL) of 17-α-hydroxyprogesterone, progesterone, estrogen, hydrocortisone, and cholesterol, and blocked with 1% BSA+HSA. 100 μl of sera from three different patients with high IgG, IgM or IgE antibodies against 17-α-hydroxyprogesterone was added to all wells. After shaking and incubation for 1 hr at room temperature, addition of enzyme labeled anti-human IgG, IgM or IgE, repeat of incubation, washing, and addition of substrate, color development was measured at 405 nm. Results were calculated as percentage of anti-17-α-hydroxyprogesterone binding to 17-α-hydroxyprogesterone and other hormones. Also, anti-estrogen binding to estrogen and other hormones in solid phase by progesterone in liquid phase was performed and calculated in a similar manner.

### Example 11

### Inter- and Intra-Assay Precision

The inter-assay reproducibility was determined by assaying eight different samples in duplicate using the hormone antibody ELISA assay on each 5 consecutive days. Each assay was performed using freshly prepared reagents. The % C.V. for samples with high O.D. (2.0 or greater) was between 5-8%, and for the samples with optical densities of 0.5-1.0 was between 10-20%.

The intra-assay reproducibility was determined by assaying eight different samples, eight different times simultaneously. Each assay was performed using freshly prepared reagents. The % C.V. for samples with O.D. between 1.0-2.5 was less than 10%, and for the samples with optical densities of 0.5-1.0 was between 12-20%.

### Example 12

### IgG and IgM Against Progesterone

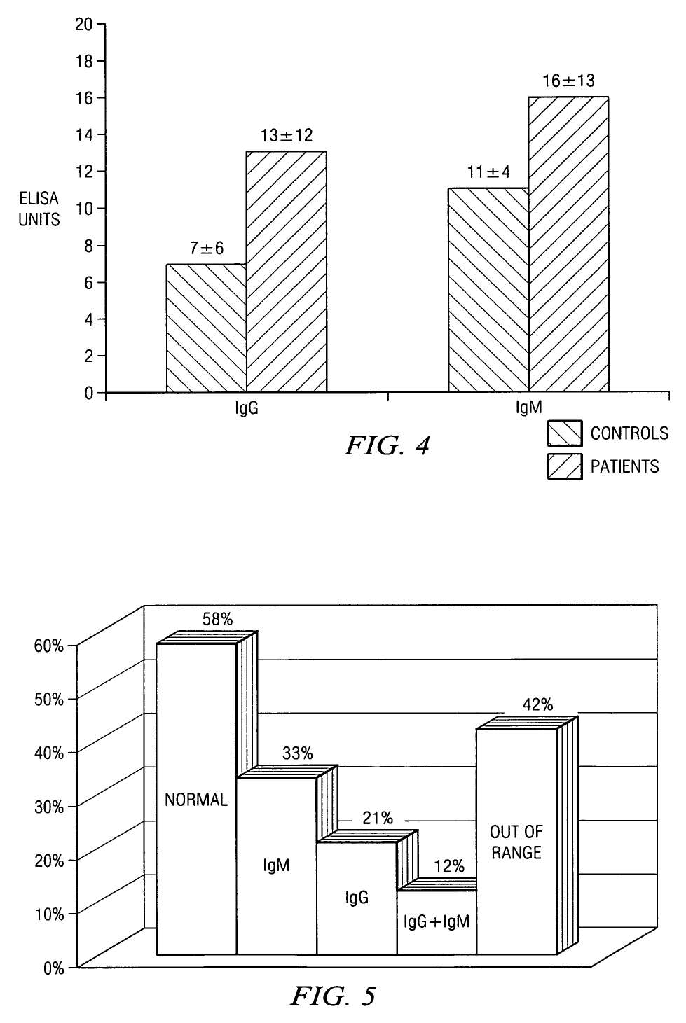

IgG, IgM and IgE antibodies against progesterone were measured in blood samples from 288 healthy females between the ages of 42-55 in the reference laboratory. Data presented in FIG. 4 shows that at a serum dilution of 1:100, ELISA values were 7±6 for IgG and 11±4 for IgM. At two standard deviations above the mean ELISA values, the upper limit of normal was determined to be 21. Based on these reference ranges 270 patients were tested for IgG and IgM. Out of these, 113 had high levels (>21 ELISA units) of IgG, IgM or both, when compared to 288 controls tested at Immunosciences Lab. Among patients with out-of-range high for both IgG and IgM (mean±S.D.), scores were 30±9.3 for IgM and 26.9±6.5 for IgG. Where IgG was out of range but not IgM, the average IgG score was 25.8±6.3, and where things were the converse, with IgM out of range but not IgG, the IgM score was 25.8±4.8. It is interesting to note that the average scores for the group where both IgM and IgG were out of range are both larger than the corresponding average for out of range groups for IgG only or IgM only. The overall mean±S.D. of 270 patients and their comparison to 288 controls are shown in FIG. 4. In comparison to the control group with and IgG of 7±6 and IgM of 11±4 ELISA units, the patients' group had mean±S.D. of ELISA units of 13±12 for IgG and 16±13 for IgM. These differences between the two groups were statistically significant (p<0.001).

Also, from the out-of-range patients, overall, at a cutoff point of 21 ELISA units, 42% of patients showed IgG, IgM or combinations of IgG and IgM antibodies (FIG. 5), the aggregate of high IgG was 21% compared to the aggregate high IgM of 33%, and combinations of IgG and IgM 12%. It seems that while the responses of IgG and IgM are not identical, they may bear subtle similarities.

### Example 13

### IgE Against Progesterone

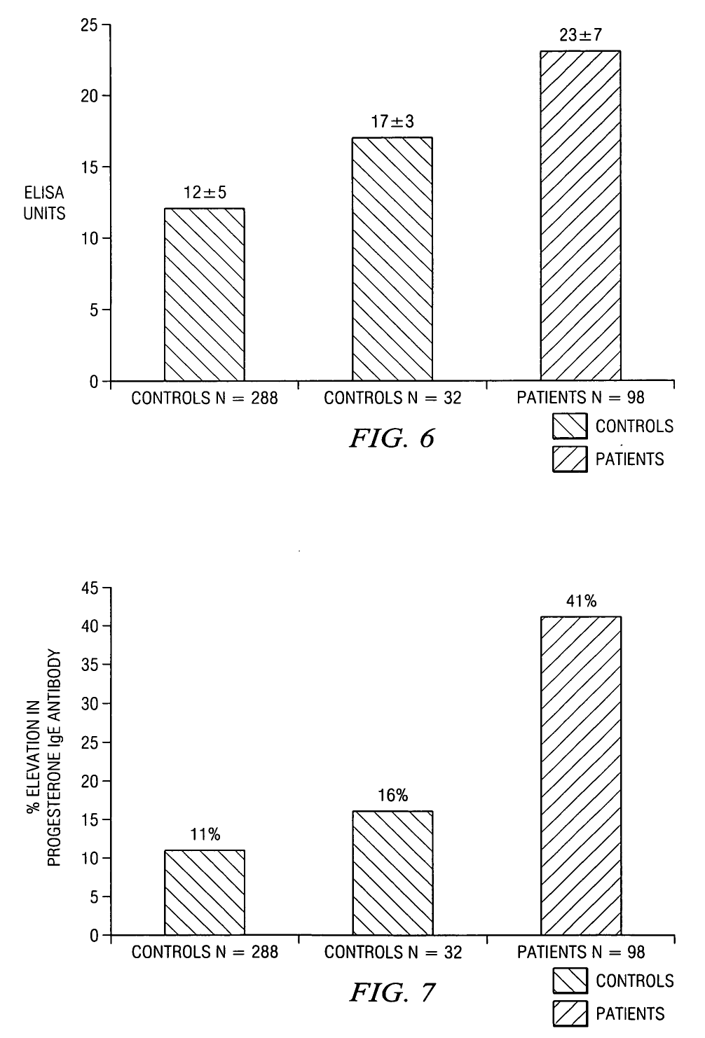

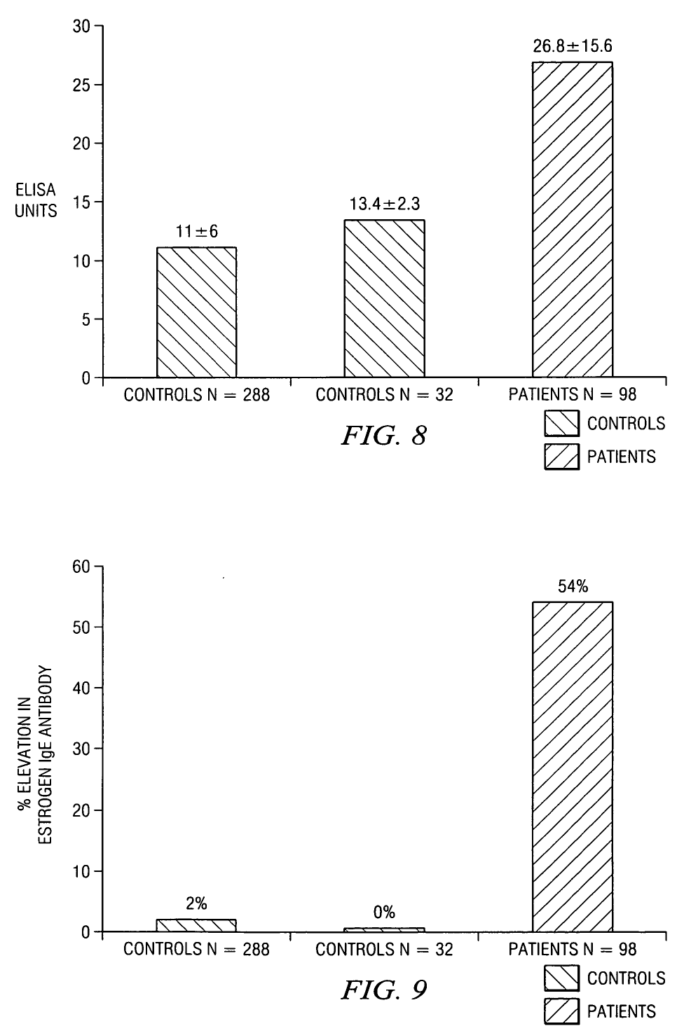

The same 288 healthy female subjects were tested for IgE antibodies against progesterone; the mean±S.D. was found to be 12±5 ELISA units. Furthermore, sera from 32 healthy subjects from the same geographic area were tested, and the mean±S.D. was 17.3±3. In comparison, the IgE antibody level against progesterone in sera from 98 patients was 23.3±7.1 (FIG. 6). Student's t-one tailed test gave highly significant differences between patients and controls (p<0.0001). However, while overall 40 out of 98 (41%) patients showed progesterone IgE levels of greater than 22 ELISA units, 58 or 59% had IgE levels lower than 23 ELISA units (FIG. 7).

### Example 14

### IgE Against Estrogen

Sera from 288 and 32 healthy female subjects and 98 patients were analyzed using ELISA assay for IgE against estrogen. The mean±S.D. for healthy groups was 11±6 and 13.4±2.3. In sera from the 80 patients, the mean±S.D. for IgE antibody against estrogen was 26.8±15.6. Student's t-one tailed test gave highly significant differences between patients versus controls (p<0.0001)

At 2 S.D. above the mean (or 23 ELISA units), 6 out of 288 (2%) and 0 out of the 32 control groups had estrogen IgE of greater than 23 ELISA units. When the same cutoff point was applied to the 98 patients' sera, 53 (54%) showed significant elevation in IgE anti-estrogen (FIG. 9). In 7 different individuals, ELISA values as high as 50-60 ELISA units were detected.

In spite of these highly significant differences between healthy and clinical populations of subjects, there are notable opportunities for IgE normal vs. clinical symptomatic misclassifications for both hormone antigens. Although all estrogen controls were within normal range, 45 out of 98 patients were within normal range. For progesterone, 4 of 32 control individuals were marginally above the normal range. Similarly, 58 of 98 patients overlapped normal range but were predominantly greater than one Standard Deviation above the mean. While the difference between two population means between control and patients was striking, there was no unequivocal boundary between “normal” vs. “abnormal” levels of IgE for either estrogen or progesterone.

### Example 15

### Examination of Specificity of Estrogen and Progesterone Antibodies by Inhibition Studies

To examine whether antibodies to estrogen or progesterone are specific or cross-reactive, we performed competition ELISA by adding specific and non-specific antigens in liquid phase and examined prevention of serum antibody binding to the antigen in solid phase. Results summarized in Table 3 show that BSA, HSA, MBP and estrogen alone did not significantly absorb the serum IgG and IgE antibodies when they were added to the liquid phase. But addition of estrogen or progesterone-BSA significantly absorbed the IgG and IgE antibodies. This inhibition of anti-estrogen binding to estrogen by estrogen-BSA in liquid phase was between 52-67%, and by progesterone-BSA was between 41-52%. For IgE anti-estrogen, this inhibition by estrogen-BSA was between 54-62%, and with progesterone-BSA, from 37-43%. These results indicate that while antibodies against hormones are specific, they may be cross-reacting between estrogen and progesterone. This cross-reaction between estrogen and progesterone antibodies may be due to structural similarities between these two hormones. Similar results were obtained when progesterone antibodies were absorbed with estrogen or progesterone bound to BSA.

TABLE 3
Level of anti-estrogen in serum and inhibition with different specific and non-
specific antigens

| IgG level (% inhibition after absorption | | | | | | | | IgE level (% inhibition after absorption | | | | | | |
|---|---|---|---|---|---|---|---|---|---|---|---|---|---|---|
| with 250 μg/mL | | | | | | | | with 250 μg/mL | | | | | | |
| | Before | | | | Es- | Estro- | Proges- | Before | | | | | | Proges- |
| Sam- | absorp- | | | | tro- | gen- | terone- | absorp- | | | | | Estrogen- | terone- |
| ple | tion | BSA | HSA | MBP | gen | BSA | BSA | tion | BSA | HSA | MBP | Estrogen | BSA | BSA |
| 1 | 1.83 | 1.79 | 1.81 | 1.67 | 1.53 | 0.61 | 0.87 | 2.15 | 2.24 | 2.11 | 2.05 | 1.70 | 0.96 | 1.35 |
| % | — | (NS) | (NS) | (NS) | (NS) | (67%) | (52%) | — | (NS) | (NS) | (NS) | (NS) | (55%) | (37%) |
| in- | | | | | | | | | | | | | | |
| hibi- | | | | | | | | | | | | | | |
| tion | | | | | | | | | | | | | | |
| 2 | 1.25 | 1.13 | 1.19 | 1.13 | 0.95 | 0.52 | 0.69 | 1.66 | 1.53 | 1.59 | 1.73 | 1.30 | 0.77 | 0.98 |
| % | — | (NS) | (NS) | (NS) | (NS) | (59%) | (45%) | — | (NS) | (NS) | (NS) | (NS) | (54%) | (41%) |
| in- | | | | | | | | | | | | | | |
| hibi- | | | | | | | | | | | | | | |
| tion | | | | | | | | | | | | | | |
| 3 | 0.76 | 0.81 | 0.78 | 0.68 | 0.54 | 0.37 | 0.45 | 1.34 | 1.26 | 1.21 | 1.11 | 0.86 | 0.51 | 0.76 |
| % | — | (NS) | (NS) | (NS) | (NS) | (52%) | (41%) | — | (NS) | (NS) | (NS) | (NS) | (62%) | (43%) |
| in- | | | | | | | | | | | | | | |
| hibi- | | | | | | | | | | | | | | |
| tion | | | | | | | | | | | | | | |

NS = non-significant

### Example 16

### Reaction of Progesterone- and Estrogen-Binding Antibodies to Different Hormones and Cholesterol

Human sera containing 17-α-hydroxyprogesterone IgG-, IgM- and IgE-binding antibodies were reacted against progesterone, estrogen, hydrocortisone and cholesterol. In comparison to 100% binding of anti-17-α-hydroxyprogesterone-binding to 17-α-hydroxyprogesterone, the IgG anti-17-α-hydroxyprogesterone-binding to progesterone, estrogen, hydrocortisone and cholesterol was 69-77%, 25-30%, 39-53%, and less than 10% respectively (Table 4). The % binding of serum IgM and IgE anti-17-α-hydroxyprogesterone to 17-1-hydroxyprogesterone, progesterone, estrogen, hydrocortisone and cholesterol was similar to the binding of IgG. This binding of anti-17-α-hydroxyprogesterone to progesterone was the highest, followed by hydrocortisone, then estrogen, to almost no binding with cholesterol. Similar to this, human sera with IgG, IgM and IgE binding antibodies against estrogen were reacted with progesterone, 17-α-hydroxyprogesterone, hydrocortisone and cholesterol, in the presence of estrogen. Results depicted in Table 5 show that the highest IgG, IgM and IgE anti-estrogen binding was first with estrogen (100%), then 17-α-hydroxyprogesterone, followed by progesterone. This binding of anti-estrogen antibodies to hydrocortisone and cholesterol was not significant (Table 5).

TABLE 4
Binding of 17-α-hydroxyprogesterone antibodies to different
hormones

| Sample # | Antibodies | HP | PR | ER | HC | C |
|---|---|---|---|---|---|---|
| 1 | IgG | 1.6 | 1.1 | 0.48 | 0.69 | 0.21 |
| | % reaction | −100 | −69 | −30 | −43 | −13 |
| | IgM | 1.3 | 0.8 | 0.4 | 0.47 | 0.18 |
| | % reaction | −100 | −61 | −31 | −36 | (NS) |
| | IgE | 1.45 | 0.94 | 0.42 | 0.58 | 0.2 |
| | % reaction | −100 | −65 | −29 | −40 | (NS) |
| 2 | IgG | 0.95 | 0.73 | 0.24 | 0.37 | 0.12 |
| | % reaction | −100 | −77 | −25 | −39 | (NS) |
| | IgM | 1.2 | 0.79 | 0.41 | 0.5 | 0.19 |
| | % reaction | −100 | −77 | −25 | −39 | (NS) |
| | IgE | 2.4 | 1.7 | 0.75 | 1.2 | 0.23 |
| | % reaction | −100 | −71 | −31 | −50 | −10 |
| 3 | IgG | 0.68 | 0.49 | 0.18 | 0.36 | 0.17 |
| | % reaction | −100 | −72 | (NS) | −53 | (NS) |
| | IgM | 0.92 | 0.72 | 0.29 | 0.51 | 0.15 |
| | % reaction | −100 | −78 | −32 | −55 | (NS) |
| | IgE | 1.1 | 0.82 | 0.37 | 0.68 | 0.18 |
| | % reaction | −100 | −77 | −25 | −39 | (NS) |

Specificity of IgG, IgM and IgE anti-17-α-hydroxyprogesterone binding to l7-α-hydroxyprogesterone, progesterone, estrogen, hydrocortisone, and cholesterol in solid phase tested by ELISA assay in the sera of three patients with possible hormone allergy. Estrogen (ER), Progesterone (PR), 17-α-hydroxy-progesterone (HP), Hydrocortisone (HC), Cholesterol (C).

TABLE 5
Binding of estrogen antibodies to different hormone

| Sample # | Antibodies | ER | PR | HP | HC | C |
|---|---|---|---|---|---|---|
| 1 | IgG | 1.2 | 0.35 | 0.42 | 0.15 | 0.12 |
| | % reaction | −100 | −29 | −35 | (NS) | (NS) |
| | IgM | 0.9 | 0.26 | 0.34 | 0.16 | 0.13 |
| | % reaction | −100 | −29 | −38 | (NS) | (NS) |
| | IgE | 0.8 | 0.21 | 0.22 | 0.17 | 0.15 |
| | % reaction | −100 | −26 | −28 | (NS) | (NS) |
| 2 | IgG | 1.8 | 0.48 | 0.75 | 0.31 | 0.19 |
| | % reaction | −100 | −27 | −42 | −17 | (NS) |
| | IgM | 2.1 | 0.65 | 0.88 | 0.44 | 0.25 |
| | % reaction | −100 | −31 | −42 | −21 | −12 |
| | IgE | 1.5 | 0.53 | 0.65 | 0.29 | 0.21 |
| | % reaction | −100 | −35 | −43 | −19 | −14 |
| 3 | IgG | 0.92 | 0.32 | 0.52 | 0.18 | 0.15 |
| | % reaction | −100 | −35 | −57 | (NS) | (NS) |
| | IgM | 1.3 | 0.44 | 0.59 | 0.23 | 0.24 |
| | % reaction | −100 | −34 | −45 | −18 | −18 |
| | IgE | 1.6 | 0.62 | 0.73 | 0.25 | 0.28 |
| | % reaction | −100 | −39 | −46 | −16 | −17 |

Specificity of IgG, IgM and IgE anti-estrogen binding to estrogen, 17-α-progesterone, progesterone, hydrocortisone, and cholesterol in solid phase in solid phase tested by ELISA assay in the sera of three patients with possible hormone allergy. Estrogen (ER), Progesterone (PR), 17-α-hydroxy-progesterone (HP), Hydrocortisone (HC), Cholesterol (C).

Without being limited to any particular mechanism of action, the foregoing data show that IgG, IgM, and/or IgE may be produced against different steroid hormones.

Although the present disclosure and its advantages have been described in detail, it should be understood that various changes, substitutions and alterations can be made herein without departing from the spirit and scope of the disclosure as defined by the following claims.

As will be understood by those of ordinary skill in the art, other equivalent or alternative methods, and/or compositions for diagnosing and/or treating a hormone allergy may be envisioned without departing from the essential characteristics thereof. For example, methods and dosages may be scaled to diagnose and/or treat subjects of different sizes (e.g., children and adults), subjects with additional allergies or conditions, and/or subjects having varying severity of allergy and/or symptoms. In addition, methods and dosages may be adapted to fluctuations over time (e.g., monthly or seasonal). These equivalents and alternatives along with obvious changes and modifications are intended to be included within the scope of the present disclosure. For example, values and/or range endpoints provided are not intended to be rigid limits for all embodiments. Moreover, one of ordinary skill in the art will appreciate that no single embodiment, use, and/or advantage is intended to universally control or exclude other embodiments, uses, and/or advantages. For example, a medical practitioner may deem circumstances to warrant giving preference to one over another. Accordingly, the foregoing disclosure is intended to be illustrative, but not limiting, of the scope of the disclosure as illustrated by the following claims.

What is claimed is:

1. A method of treating a hormone allergy comprising administering to a subject having a hormone allergy 0.5 mg of progesterone intradermally or sublingually.

2. The method of claim 1, further comprising treating a symptom or disorder related to the hormone allergy.

3. The method of claim 1, further comprising treating a symptom or disorder selected from the group consisting of: irritation or pain in the neck, nose, eyes, throat, ear, skin, back, hips, upper extremities and lower extremities, headache, including migraine headaches, shortness of breath, including asthma, arthritis, infertility, fibromyalgia, chronic fatigue syndrome, multiple sclerosis, and any combination thereof.

4. A method of treating a hormone allergy comprising administering to a subject having a hormone allergy 0.4 mg of estrogen intradermally or sublingually.

5. The method of claim 4, further comprising treating a symptom or disorder related to the hormone allergy.

6. The method of claim 4, further comprising treating a symptom or disorder selected from the group consisting of: irritation or pain in the neck, nose, eyes, throat, ear, skin, back, hips, upper extremities and lower extremities, headache, including migraine headaches, shortness of breath, including asthma, arthritis, infertility, fibromyalgia, chronic fatigue syndrome, multiple sclerosis, and any combination thereof.

7. The method of claim 6, wherein the subject experiences relief of at least one symptom in less than about 30 minutes.

8. A method of diagnosing a hormone allergy in a subject comprising:
determining the level of an anti-hormone antibody in blood serum of the subject; and
comparing the level of the anti-hormone antibody in the blood serum of the subject to the normal level of an anti-hormone antibody in the blood sera of individuals who do not have a hormone allergy,
wherein a statistically significant elevated level of anti-hormone antibody in the blood serum of the subject as compared to the normal level of anti-hormone antibody in the blood sera of individuals who do not have hormone allergy is indicative of hormone allergy in the subject.

9. The method of claim 8, wherein the anti-hormone antibody comprises an anti-progesterone antibody.

10. The method of claim 8, wherein the anti-hormone antibody comprises an anti-estrogen antibody.

11. The method of claim 8, wherein the anti-hormone antibody comprises an IgE antibody.

12. The method of claim 8, wherein determining comprises using an ELISA assay.

13. A method of treating an IgE-mediated reaction to a bodily substance in a human comprising administering an anti-IgE treatment to the human.

14. The method of claim 13, wherein the IgE-mediated reaction further comprises a hormone allergy.

15. The method of claim 14, wherein the hormone allergy further comprises a progesterone or estrogen allergy.

16. The method of claim 13, wherein the IgE-mediated reaction further comprises a disease or disorder selected from the group consisting of: migraine headaches, arthritis, infertility, fibromyalgia, chronic fatigue syndrome, multiple sclerosis, and any combination thereof.

17. The method of claim 13, wherein the IgE-mediated reaction further comprises at least one symptom selected from the group consisting of: pain in the neck, nose, eyes, throat, ear, skin, back, hips, upper extremities and lower extremities, headache, shortness of breath, and any combination thereof.

18. The method of claim 13, wherein the treatment further comprises an anti-IgE antibody or fragment or derivative thereof.

19. The method of claim 18, wherein the antibody further comprises a humanized antibody.

20. The method of claim 19, wherein the antibody further comprises Xolair or TNX-901.

21. A composition for attenuating an IgE-mediated hormone immune response in a subject having a hormone allergy comprising:
about 10⁻¹⁰ milligrams to about 10⁻¹ milligrams of the hormone; and
a pharmaceutically acceptable additive.

22. The composition of claim 21, wherein the additive is selected from the group consisting of magnesium stearate, cellulose, calcium carbonate, starch-gelatin paste, talc, carrier, excipient, and diluent.
